(19) United States
(12) Patent Application Publication
(10) Pub. No.: US 2023/0160839 A1
(43) Pub. Date: May 25, 2023

(54) RADIATION IMAGING APPARATUS

(71) Applicant: CANON KABUSHIKI KAISHA, Tokyo (JP)

(72) Inventor: Hiroaki Niwa, Kanagawa (JP)

(21) Appl. No.: 18/057,626

(22) Filed: Nov. 21, 2022

(30) Foreign Application Priority Data
Nov. 25, 2021 (JP) 2021-191525

(51) Int. Cl. G01N 23/04; G01N 23/06

(52) U.S. Cl. CPC G01N 23/04; G01N 23/06; G01N 2223/04; G01N 2223/40; G01N 2223/628

(57) ABSTRACT

A radiation source applies radiation to a subject. A radiation generation apparatus controls the radiation source. A radiation imaging apparatus includes a pixel array including a plurality of image signal output pixels that outputs image signals based on the radiation applied from the radiation source and a plurality of dose detection pixels that detects a dose based on the radiation applied from the radiation source, and includes a control unit that controls driving of the radiation imaging apparatus and a radiation irradiation timing. The control unit includes a prediction unit that predicts an irradiation time from a result of detection of an integrated irradiation amount by the dose detection pixels, and a drive control unit that changes at least one of the number of frames to be captured and an offset correction processing method based on the prediction result.

### BACKGROUND

### Field of the Disclosure

The present disclosure relates to a radiation imaging apparatus.

### Description of the Related Art

As imaging apparatuses for medical image diagnosis and non-destructive inspection using radiation, a detection apparatus and a radiation detection apparatus that use a matrix substrate including a pixel array in which switch elements such as thin-film transistors (TFTs) and conversion elements such as photoelectric conversion elements are used in combination are in practical use. For example, in the medical image diagnosis, such a radiation imaging apparatus is used to capture a still image in general radiography and to capture a moving image in fluorography. In the non-destructive inspection, such a radiation imaging apparatus is used to inspect a state of piping and welding. In the non-destructive inspection, to identify a state of a subject, radiation irradiation is performed for a long time.

In recent years, multifunctionality of such a detection apparatus has been studied. As one of the studies, incorporation of a function enabling the detection apparatus to obtain irradiation information while a radiation source performs radiation irradiation has been studied. Examples of the function include a function of identifying an exposure start timing at which the radiation source starts the radiation irradiation, and a function of identifying a radiation dose and an integrated irradiation amount. This function also enables the detection apparatus to monitor the integrated irradiation amount and control the radiation source to end the irradiation when the integrated irradiation amount reaches a correct amount.

Such a function is called automatic exposure control (AEC).

Japanese Patent Application No. 2011-249591 discusses a radiation imaging apparatus that acquires radiation dose information by reading out charge accumulated in pixels during radiation irradiation and gives an instruction to end the radiation irradiation based on the acquired radiation dose information and a target dose value.

The applicant of the present disclosure has found that the radiation imaging apparatus that determines whether to end the radiation irradiation based on the radiation dose information and the target dose value, such as the apparatus discussed in Japanese Patent Application No. 2011-249591, has improvements to improve captured image quality.

### SUMMARY OF THE DISCLOSURE

The present disclosure is directed to a new radiation imaging apparatus with improved captured image quality or excellent usability.

According to an aspect of the present disclosure, a radiation imaging apparatus includes an element portion including a first element and a second element each configured to output an electric signal based on charge accumulated in response to radiation irradiation, a readout unit configured to read out the electric signal, and a control unit configured to control an operation of the readout unit so that the readout unit performs a dose detection operation of reading out the electric signal from the second element during the radiation irradiation, and performs an image readout operation of reading out the electric signal from the first element after stop of the radiation irradiation. The control unit includes an accumulation time acquisition unit configured to acquire information about an accumulation time of the charge based on the electric signal read out by the dose detection operation, and determines, based on the information about the accumulation time, whether to cause the readout unit to perform, following the image readout operation, a subsequent correction value acquisition operation for correcting the electric signal read out by the image readout operation during the stop of the radiation irradiation.

According to another aspect of the present disclosure, a radiation imaging apparatus includes an element portion including a first element and a second element each configured to output an electric signal based on charge accumulated in response to radiation irradiation, a readout unit configured to read out the electric signal, and a control unit configured to control an operation of the readout unit so that the readout unit performs an image readout operation of reading out the electric signal from the first element, and performs a dose detection operation of reading out the electric signal from the second element during the radiation irradiation. The control unit includes an accumulation time acquisition unit configured to acquire information about an accumulation time of the charge based on the electric signal read out by the dose detection operation, and determines a number of times of the image readout operation based on the information about the accumulation time.

According to yet another aspect of the present disclosure, a radiation imaging apparatus includes an element portion including a first element and a second element each configured to output an electric signal based on charge accumulated in response to radiation irradiation, a readout unit configured to read out the electric signal, and a control unit configured to control an operation of the readout unit so that the readout unit performs a first operation of reading out the electric signal from the second element during the radiation irradiation, performs a second operation of reading out the electric signal from the first element following the first operation, and performs a third operation of reading out the electric signal from the first element during stop of the radiation irradiation following the second operation. The control unit includes an irradiation time acquisition unit configured to acquire information about an irradiation time of the radiation based on the electric signal read out by the first operation, and determines whether to cause the readout unit to perform the third operation based on the information about the irradiation time.

Further features of the present disclosure will become apparent from the following description of exemplary embodiments with reference to the attached drawings.

### BRIEF DESCRIPTION OF THE DRAWINGS

FIG. 1 is an overall diagram of a radiation imaging system, according to one or more embodiment of the subject disclosure.

FIG. 2 is a configuration diagram of a detection unit, according to one or more embodiment of the subject disclosure.

FIG. 3 is a configuration diagram of a control unit, according to one or more embodiment of the subject disclosure.

FIG. 4 is a timing chart at the time of automatic exposure control (AEC), according to one or more embodiment of the subject disclosure.

FIG. 5 is a flowchart illustrating a first exemplary embodiment of the present disclosure.

FIG. 6 is a flowchart illustrating a second exemplary embodiment of the present disclosure.

FIG. 7 is a diagram illustrating an irradiation time prediction operation according to an exemplary embodiment of the present disclosure.

### DESCRIPTION OF THE EMBODIMENTS

Exemplary embodiments of the present disclosure will be described below with reference to the drawings.

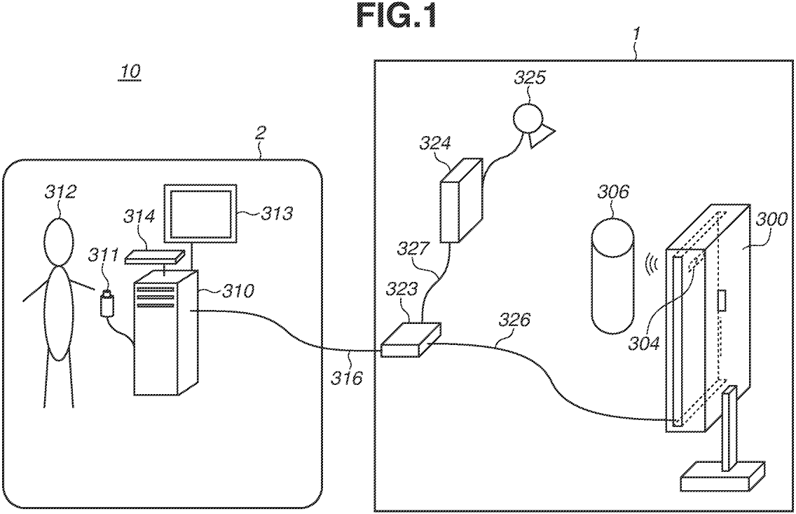

FIG. 1 is an overall diagram of a radiation imaging system 10 for non-destructive inspection according to a first exemplary embodiment. The radiation imaging system 10 includes a radiation imaging apparatus 300, a communication control apparatus 323, a radiation generation apparatus 324, a radiation source 325, an imaging apparatus cable 326, and a radiation generation apparatus communication cable 327. The radiation imaging apparatus 300, the communication control apparatus 323, the radiation generation apparatus 324, the radiation source 325, the imaging apparatus cable 326, and the radiation generation apparatus communication cable 327 are disposed in an inspection room 1.

The radiation imaging system 10 further includes a control apparatus 310, a radiation irradiation switch 311, a display apparatus 313, an input apparatus 314, and a communication cable 316. The control apparatus 310, the radiation irradiation switch 311, the display apparatus 313, the input apparatus 314, and the communication cable 316 are disposed in a control room 2.

The radiation imaging apparatus 300 detects radiation having passed through a subject 306, such as a pipe, to generate radiation image data. The communication control apparatus 323 performs control to enable the radiation generation apparatus 324 and the control apparatus 310 to communicate with each other. The radiation generation apparatus 324 controls the radiation source 325 to irradiate the subject 306 with radiation based on irradiation conditions. The radiation source 325 irradiates the subject 306 with the radiation under the control of the radiation generation apparatus 324.

The imaging apparatus cable 326 is used to connect the radiation imaging apparatus 300 and the communication control apparatus 323. The radiation generation apparatus communication cable 327 is used to connect the radiation generation apparatus 324 and the communication control apparatus 323.

The control apparatus 310 controls the entire radiation imaging system 10 by communicating with the radiation generation apparatus 324 and the radiation imaging apparatus 300 via the communication control apparatus 323. The radiation irradiation switch 311 is operated by an operator 312 to input a radiation irradiation timing. The input apparatus 314 is used to input an instruction from the operator 312, and various input devices such as a keyboard and a touch panel are used for the input apparatus 314. The display apparatus 313 displays radiation image data subjected to image processing, a graphical user interface (GUI), or the like, using a display or the like.

Next, an operation of the radiation imaging system 10 will be described. First, the control apparatus 310 sets imaging information based on an operation by the operator 312. Alternatively, the control apparatus 310 may set the imaging information by selecting a preset imaging protocol. Further, based on the set information, the control apparatus 310 determines imaging conditions, such as a tube voltage and a tube current, for use in the radiation irradiation.

After imaging preparations are completed, the operator 312 presses the radiation irradiation switch 311. When the radiation irradiation switch 311 is pressed, the radiation imaging apparatus 300 performs preparations, and then the radiation source 325 irradiates the subject 306 with radiation. The radiation imaging apparatus 300 communicates with the radiation generation apparatus 324 to control the start and end of the radiation irradiation. The radiation applied to the subject 306 passes through the subject 306, and enters the radiation imaging apparatus 300. The radiation imaging apparatus 300 converts the incident radiation into visible light and then detects the light as radiation image signals using photoelectric conversion elements.

The radiation imaging apparatus 300 drives the photoelectric conversion elements to read out the radiation image signals, and converts the analog signals into digital signals using an analog-to-digital (AD) conversion circuit to acquire digital radiation image data. The acquired digital radiation image data is transferred from the radiation imaging apparatus 300 to the control apparatus 310. The control apparatus 310 performs image processing on the received digital radiation image data. The control apparatus 310 displays a radiation image based on the processed radiation image data, on the display apparatus 313. The control apparatus 310 functions as an image processing apparatus and a display control apparatus. The above is the description of the operation of the radiation imaging system 10.

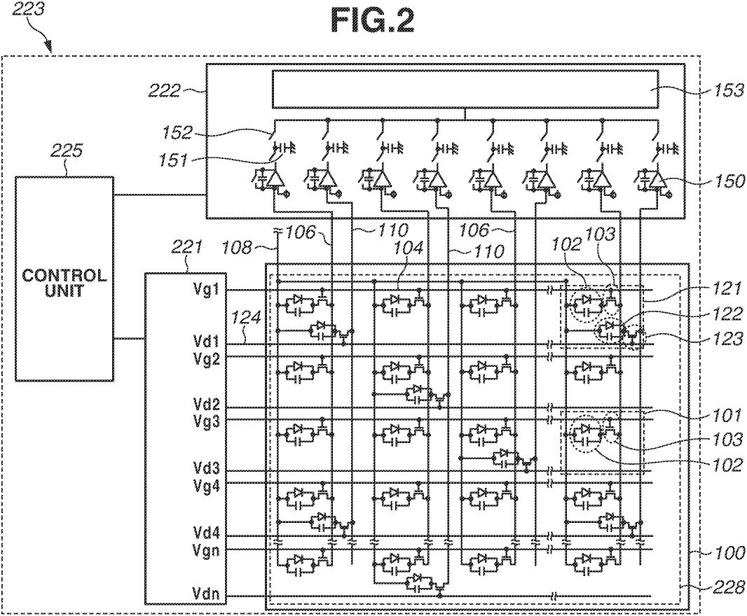

FIG. 2 is a configuration diagram of a detection unit 223 included in the radiation imaging apparatus 300. The detection unit 223 includes a support substrate 100, a drive circuit 221, a readout circuit 222, and a control unit 225.

A pixel array 228 is disposed on the support substrate 100. The pixel array 228 is an example of an imaging area. The pixel array 228 includes a plurality of pixels arranged in a matrix. The pixel array 228 includes first pixels 101 and second pixels 121. The first pixels 101 and the second pixels 121 will be described next.

To acquire a radiation image, each of the first pixels 101 includes a conversion element 102 that converts incident radiation or incident light into charge corresponding to an amount thereof, and a switch element 103 that outputs the charge generated by the conversion element 102 to a corresponding one of signal lines 106. The conversion element 102 is an indirect conversion element using, for example, a scintillator that converts radiation into light, and a photoelectric conversion element that converts the light generated by the scintillator into charge. In the case of the above configuration, the conversion element 102 and the switch element 103 forms a first element capable of outputting an electric signal based on the charge accumulated in response to the radiation irradiation. Alternatively, as the conversion element 102, for example, a direct conversion element that directly converts radiation into charge is usable. As the switch element 103, for example, a thin-film transistor (TFT) using amorphous silicon or polycrystalline silicon is usable. For example, polycrystalline silicon may be used for the switch element 103 depending on desired characteristics of the TFT. Further, a semiconductor material for the TFT is not limited to silicon, and other semiconductor materials such as germanium and a compound semiconductor may be used.

The conversion element 102 includes a first electrode electrically connected to a first main electrode of the switch element 103, and a second electrode electrically connected to a corresponding one of bias lines 108. Each of the bias lines 108 is commonly connected to the second electrodes of the plurality of conversion elements 102 arranged along the corresponding column.

A common bias voltage is supplied to the bias lines 108 provided for the respective columns. The bias lines 108 receive the bias voltage supplied from a power supply circuit (not illustrated).

The switch elements 103 includes a second main electrode electrically connected to a corresponding one of the signal lines 106. The second main electrodes of the switch elements 103 of the pixels arranged along each column are commonly connected to a corresponding one of the signal lines 106. The signal lines 106 are provided for respective columns of the pixels. The signal lines 106 are electrically connected to the readout circuit 222. Drive lines 104 are electrically connected to control electrodes of the switch elements 103. Each of the drive lines 104 is commonly connected to the control electrodes of the switch elements 103 of the plurality of first pixels 101 arranged along the corresponding row. Gate control voltages Vg1 to Vgn are applied from the drive circuit 221 to the respective drive lines 104.

Each of the second pixels 121 includes a detection element 122, and a switch element 123 that outputs charge generated by the detection element 122 to a corresponding one of detection lines 110. The detection element 122 converts incident radiation or incident light into charge corresponding to an amount thereof in order to obtain the total dose of radiation incident during the radiation irradiation. The switch element 123 outputs the charge generated by the detection element 122 to a corresponding one of the detection lines 110. In the case of the above configuration, similarly to the conversion element 102 and the switch element 103 described above, the detection element 122 and the switch element 123 forms a second element capable of outputting an electric signal based on the charge accumulated in response to the radiation irradiation. The first element formed by the conversion element 102 and the switch element 103 and the second element formed by the detection element 122 and the switch element 123 form an element portion. Each of the second pixels 121 further includes the conversion element 102 and the switch element 103. The conversion element 102 and the switch element 103 in each of the second pixels 121 respectively operate in a manner similar to the conversion element 102 and the switch element 103 in each of the first pixels 101.

The detection element 122 includes a first electrode electrically connected to a first main electrode of the switch element 123. The detection element 122 also includes a second electrode electrically connected to a corresponding one of the bias lines 108 provided for the respective columns. Second main electrodes of the switch elements 123 arranged along each column are connected to a corresponding one of the detection lines 110. The detection lines 110 are electrically connected to the readout circuit 222. Drive lines 124 provided for the respective rows are connected to control electrodes of the switch elements 123. Gate control voltages Vd1 to Vdn are applied from the drive circuit 221 to the respective drive lines 124.

As illustrated in FIG. 2, the pixel array 228 according to the present exemplary embodiment includes the plurality of second pixels 121. Alternatively, the pixel array 228 may include one second pixel 121, and the number and positions thereof are not limited to the present exemplary embodiment. In a case where the pixel array 228 includes the plurality of second pixels 121, the dose of incident radiation may be detected by one of the detection elements 122 of the plurality of second pixels 121, or may be detected by the plurality of detection elements 122. Alternatively, the pixel array 228 may include no second pixel 121. In this case, the drive lines 104 may be driven during the radiation irradiation, and the first pixels 101 may detect the total dose of incident radiation.

The readout circuit 222 is a readout unit that reads out the electric signals output from the first pixels 101 or the second pixels 121. Each of the signal lines 106 and the detection lines 110 is connected to a corresponding one of inverted input terminals of operation amplifiers 150. The inverted input terminals of the operation amplifiers 150 are respectively connected to output terminals via feedback capacitors, and non-inverted input terminals thereof are respectively connected to any fixed potentials. The operation amplifiers 150 function as charge-voltage conversion circuits. An AD converter 153 is connected to rear stages of the operation amplifiers 150 via sample-and-hold circuits 151 and multiplexers 152. The readout circuit 222 is a digital conversion circuit that converts the charge transferred from the conversion elements 102 of the first pixels 101 and the conversion elements 102 and the detection elements 122 of the second pixels 121 via the signal lines 106 and the detection lines 110, into digital electric signals. The readout circuit 222 may have a configuration in which the circuits are integrated, or may be individually provided for each of the circuits.

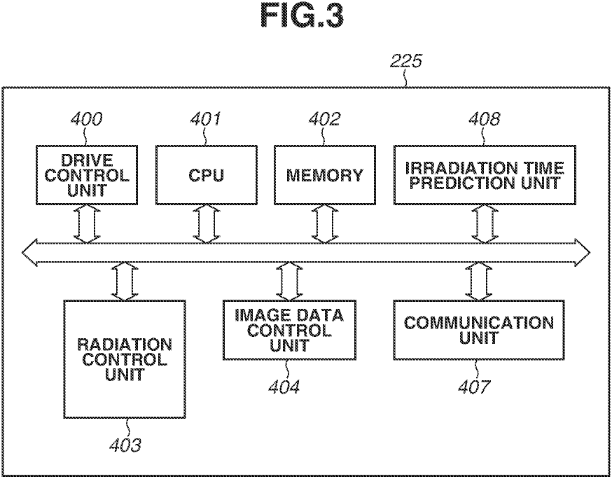

FIG. 3 is a configuration diagram of the control unit 225. The control unit 225 includes a drive control unit 400, a central processing unit (CPU) 401, a memory 402, a radiation control unit 403, an image data control unit 404, a communication unit 407, and an irradiation time prediction unit 408. The drive control unit 400 controls the drive circuit 221 and the readout circuit 222 based on information from a signal processing unit (not illustrated) and a command from the control apparatus 310. The CPU 401 controls the entire radiation imaging apparatus 300 by using programs and various kinds of data stored in the memory 402. The memory 402 stores, for example, the programs and the various kinds of data to be used when the CPU 401 performs processing. Further, the memory 402 stores various kinds of data obtained by the processing of the CPU 401, and radiation image data.

The radiation control unit 403 controls the radiation generation apparatus 324 based on information from the signal processing unit and information from the drive control unit 400. The radiation control unit 403 exchanges control-related information (e.g., a radiation irradiation start notification, a radiation irradiation end notification, and a radiation irradiation amount) with the radiation generation apparatus 324 via the communication unit 407. The image data control unit 404 saves the image data from the readout circuit 222 in the memory 402, and controls communication with the control apparatus 310. The image data control unit 404 and the control apparatus 310 exchange the radiation image data and the control-related information (e.g., a control command) with each other. The communication unit 407 communicates with an external apparatus connected thereto in a wired or wireless manner. The irradiation time prediction unit 408 functions as an accumulation time acquisition unit, and predicts an accumulation time (described below) during which the conversion elements 102 accumulate charge, based on a result of detection of an integrated irradiation amount (described below) using the dose detection pixels (the second pixels 121). In a case where the accumulation time during which the conversion elements 102 accumulate charge and a radiation irradiation time are substantially equal to each other, the accumulation time may be replaced with the irradiation time. In the present exemplary embodiment (and a second exemplary embodiment described below), to suppress wasteful radiation irradiation, the operation is controlled so that an irradiation start timing and an accumulation start timing are substantially synchronized with each other, and an irradiation end (stop) timing and an accumulation end timing are substantially synchronized with each other. Thus, the accumulation time and the irradiation time are treated to be equal to each other. Likewise, a predicted accumulation time and a predicted irradiation time are treated to be equal to each other.

Functions and processing of the control unit 225 to be described below are implemented by the CPU 401 reading out the programs stored in the memory 402 and executing the programs. Alternatively, the CPU 401 may read out programs stored in a recording medium such as a secure digital (SD) card, in place of the memory 402.

Further alternatively, at least a part of the functions and the processing of the control unit 225 may be implemented by causing, for example, a plurality of CPUs and memories to cooperate with each other. Yet further alternatively, at least a part of the functions and the processing of the control unit 225 may be implemented by using a hardware circuit.

Next, a dose control operation (automatic exposure control (AEC)) by the radiation imaging system 10 will be described. In a case where radiation imaging is performed, the operator 312 first inputs irradiation conditions, such as a dose, a maximum irradiation time, a tube current, and a tube voltage, and a radiation detection region (a region of interest (ROI)) where the radiation is to be monitored, to the control apparatus 310. The control apparatus 310 transmits the input irradiation conditions and the input radiation detection area (ROI) to the radiation imaging apparatus 300 and the radiation generation apparatus 324. Thereafter, when the imaging preparations are completed and the operator 312 presses the radiation irradiation switch 311, the radiation source 325 irradiates the subject 306 with the radiation via the radiation generation apparatus 324. The radiation passes through the subject 306 and enters the radiation imaging apparatus 300.

The AEC will be described now. The radiation imaging apparatus 300 performs the AEC based on the incident radiation. When the radiation having entered the ROI is detected by the detection elements 122, the CPU 401 of the radiation imaging apparatus 300 calculates the integrated irradiation amount which is an integrated value of the dose (the dose reaching the ROI) detected for a predetermined period. Further, the CPU 401 determines a radiation irradiation stop timing based on the integrated irradiation amount and a preset correct dose.

The radiation control unit 403 notifies the radiation generation apparatus 324 of stop of the radiation irradiation based on the determined radiation irradiation stop timing. The radiation generation apparatus 324 stops the radiation irradiation based on the radiation irradiation stop timing. In the present exemplary embodiment, the radiation imaging apparatus 300 notifies the radiation generation apparatus 324 of the stop of the radiation irradiation, but the notification is not limited thereto. The radiation imaging apparatus 300 may transmit the reaching dose detected for each predetermined time as the detection result, and the radiation generation apparatus 324 may calculate the integrated value of the reaching dose.

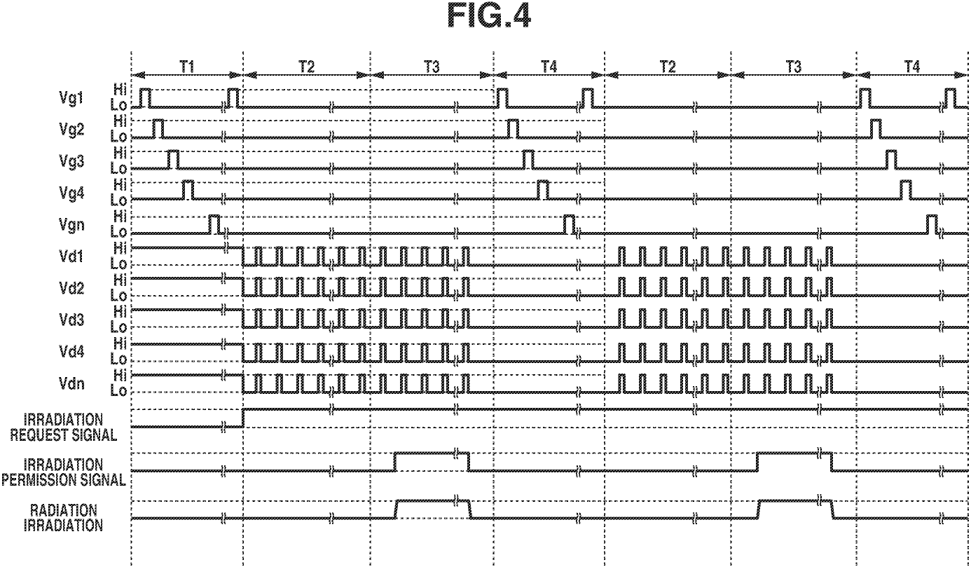

FIG. 4 is a timing chart of the AEC. A period T1 illustrated in FIG. 4 indicates an idling period during standby. During the period T1, signals applied from the drive circuit 221 causes the pixel array 228 to repeat an idling operation as illustrated in FIG. 4. The idling operation is performed, for example, until offset component acquisition is started after the detection unit 223 is powered ON. The period T1 is a time period during which the operator 312 inputs the imaging information, or a time period before the operator 312 presses the radiation irradiation switch 311.

During the period T1, to periodically remove dark currents generated from the conversion elements 102, Hi signals are periodically applied to the gate control voltages Vg1 to Vgn, and the switch elements 103 of the first pixels 101 are scanned. Likewise, to remove dark currents generated from the detection elements 122 of the second pixels 121, Hi signals are constantly applied to the gate control voltages Vd1 to Vdn, and the switch elements 123 of the second pixels 121 are put into conductive states. The Hi signals are voltages for turning ON the switch elements 103 and 123, and Lo signals are voltages (e.g., 0 V) for turning OFF the switch elements 103 and 123.

Further, the timing chart illustrated in FIG. 4 corresponds to a case where the dose of incident radiation is detected using the plurality of detection elements 122. In this case, the same target radiation dose is set for the plurality of detection elements 122. The target radiation dose is calculated by the control unit 225 based on the imaging conditions and the like. In a case where the detected value of at least one of the detection elements 122 reaches the target radiation dose, the control unit 225 outputs a radiation irradiation stop signal.

Alternatively, the control unit 225 may output the stop signal in a case where the detected values of all of the detection elements 122 reach the target radiation dose, or in a case where an average value of the detected values of all of the detection elements 122 reaches the target radiation dose. Further alternatively, different target radiation doses may be set for the plurality of detection elements 122. The target radiation dose(s) of the detection elements 122 and the condition for the control unit 225 to output the stop signal are set as appropriate based on the subject 306, the imaging conditions, the positions of the detection elements 122 in the pixel array 228, and the like.

When the radiation irradiation switch 311 is pressed, the radiation imaging apparatus 300 receives an irradiation request signal. When the radiation imaging apparatus 300 receives the irradiation request signal, the period T1 transitions to a period T2. The period T2 is a period during which the offset component acquisition is performed. During the period T2, the gate control voltages Vd1 to Vdn are turned ON in a constant cycle in a state where the radiation irradiation is not performed, and an offset component is acquired. The offset component includes crosstalk and a dark current. The number of times of turning ON the gate control voltages Vd1 to Vdn is determined in advance. The ON cycle of the gate control voltages Vd1 to Vdn is the same as a cycle of radiation signal detection during the radiation irradiation. Using the same driving cycle makes it possible to bring an offset signal amount at the time of the offset component acquisition and an offset signal amount on the detection signal during the radiation irradiation close to each other, and to correct the offset component during the radiation irradiation, with high accuracy.

After the offset component acquisition ends, the period T2 transitions to a period T3. The period T3 is a period during which the radiation irradiation is performed in order to acquire a radiation image. In the present exemplary embodiment, the radiation irradiation is actually started when the radiation generation apparatus 324 receives an irradiation permission signal from the radiation imaging apparatus 300. During the period T3, the Lo signals are applied to the gate control voltages Vg1 to Vgn for driving the switch elements 103, and the conversion elements 102 accumulate the charge corresponding to the incident radiation dose. Further, the Hi signals are applied to the gate control voltages Vd1 to Vdn for driving the switch elements 123 in a constant detection cycle, and the charge detected by the detection elements 122 is transmitted to the readout circuit 222 via the detection lines 110. The readout circuit 222 supplies electric signals based on the detected charge, to the control unit 225 via the signal processing unit (not illustrated). These series of operation correspond to a dose detection operation of reading out the electric signals from the detection elements 122 forming the second elements during the radiation irradiation. The control unit 225 acquires the radiation dose incident on the detection elements 122 in each detection cycle.

As described above, the control unit 225 is capable of controlling the operation of the readout circuit 222 serving as the readout unit. During the periods T2 and T3, the switch elements 123 are continuously driven in the same cycle. In a case where the detection cycle is changed or the detection is temporarily stopped during the transition from the period T2 to the period T3, an unintended signal fluctuation occurs due to switching of the driving. As a result, the detection signal at an initial stage of the period T3 may be affected, and detection accuracy may be deteriorated.

In FIG. 4, the gate control voltages Vd1 to Vdn applied to the control electrodes of the switch elements 123 are turned into the Hi signals at the same time, but the operation during the period T2 is not limited thereto. For example, the timings of the Hi signals of the gate control voltages Vd1 to Vdn corresponding to the switch elements 123 connected to the same detection line 110 may be made different from each other. In this case, although the signal amount readable at one time is reduced, space resolution of the detection area can be increased. Further, the offset components of the detection elements 122 are acquired in the cycle same as the driving cycle of the corresponding switch elements 123.

The charge transmitted from the detection elements 122 to the readout circuit 222 is converted into voltage information by the operation amplifiers 150. Thereafter, the voltage information is sampled by the sample-and-hold circuits 151 based on the detection cycle, and the sampled voltage information is converted into electric signals as digital data by the AD converter 153 via the multiplexers 152.

The control unit 225 uses the acquired offset components to correct the radiation dose that is obtained by converting the charge detected by the detection elements 122 into electric signals. Thereafter, the control unit 225 determines whether to stop the radiation exposure based on a cumulative value of the corrected radiation dose (the integrated irradiation amount) and the target radiation dose.

In a case where the cumulative value of the applied radiation has reached the target radiation dose or in a case where the cumulative value of the applied radiation is expected to reach the target radiation dose, the control unit 225 outputs the radiation irradiation stop signal to the radiation generation apparatus 324. The radiation generation apparatus 324 stops the radiation irradiation from the radiation source 325.

A length of the period T3 is set based on an imaging mode and the irradiation time input in advance. For example, in a case where the cumulative value does not reach the target radiation dose unlike the above-described case, but an upper limit of the irradiation time input as the irradiation information is reached, the control unit 225 performs control to stop the radiation irradiation.

After the cumulative value of the radiation dose detected by the detection elements 122 reaches the target radiation dose, or after a predetermined time elapses, the period T3 transitions to a period T4.

The period T4 is a period after the radiation irradiation, during which the captured radiation image is acquired. During the period T4, the control unit 225 outputs a control signal for reading out the signal charge accumulated in the conversion elements 102, to the drive circuit 221. The drive circuit 221 sequentially applies the Hi signals to the gate control voltages Vg1 to Vgn in response to the control signal, and sequentially scans the switch elements 103 of the first pixels 101 and the second pixels 121. The charge accumulated in the conversion elements 102 is converted into the voltage information by the operation amplifiers 150, the voltage information is sampled by the sample-and-hold circuits 151, and the sampled voltage information is converted into electric signals as digital data by the AD converter 153 via the multiplexers 152. The radiation image is formed based on the electric signals obtained by the conversion element 102 and read out therefrom. The operation during the period T4 corresponds to an image readout operation of reading out the electric signals from the conversion elements 102 forming the first elements after the stop of the radiation irradiation. In the case of imaging of a plurality of frames (described below), the operations during the periods T2 to T4 are repeated a desired number of times.

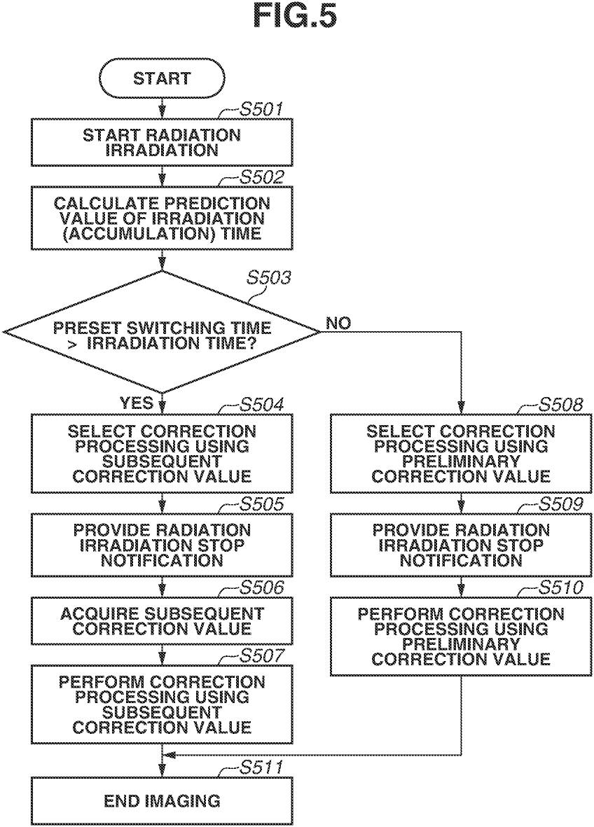

FIG. 5 is a flowchart illustrating the operations of the control unit 225 according to the present exemplary embodiment. In step S501, the radiation irradiation is started by the user’s operation. In step S502, the drive control unit 400 performs AEC driving of the radiation imaging apparatus 300 to calculate the integrated irradiation amount, which is the integrated value of the dose (the reaching dose), using the irradiation time prediction unit 408 during the imaging. The irradiation time prediction unit 408 calculates an increase in the integrated irradiation amount per constant unit time, and predicts and calculates, based on a separately set target irradiation dose, the irradiation time taken to reach the target irradiation dose, i.e., the accumulation time. In other words, in step S502, information about the charge accumulation time is acquired based on the electric signals read out by the dose detection operation.

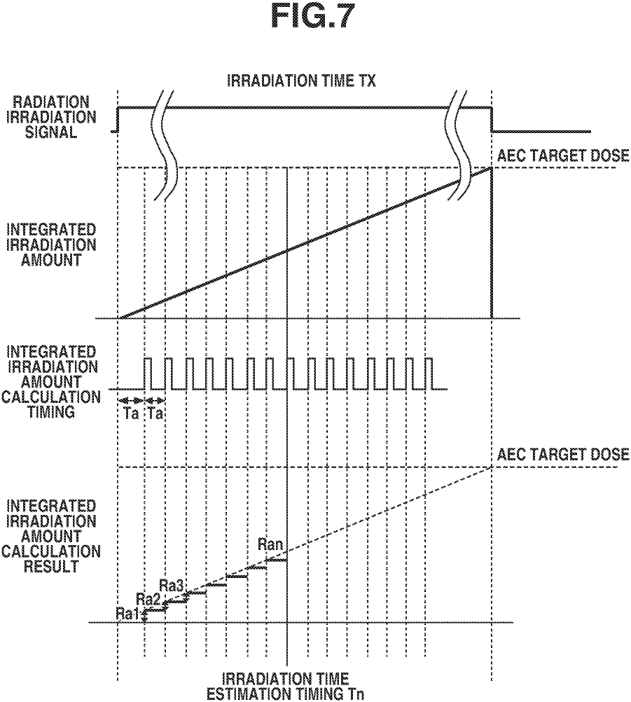

The operation of the irradiation time prediction unit 408 that predicts and calculates the irradiation time will be described with reference to FIG. 7. A horizontal axis in FIG. 7 represents an irradiation time TX. A vertical axis in FIG. 7 represents a radiation irradiation signal generated by the radiation control unit 403, the integrated irradiation amount of radiation, a calculation timing of the integrated irradiation amount in the AEC driving, and a calculation result of the integrated irradiation amount at the calculation timing.

When the radiation irradiation signal is asserted by the control of the radiation control unit 403, the radiation irradiation is actually started, and the irradiation amount applied to the subject 306 is integrated. At this time, the irradiation time prediction unit 408 monitors the irradiation amount at each constant timing (each integrated irradiation amount calculation timing) Ta in the above-described AEC driving, and adds the irradiation amount as the integrated irradiation amount. This operation is repeated, and at a desired irradiation time estimation timing Tn, the irradiation time taken to reach the preset AEC target dose is calculated based on the average value of increases Ra1 to Ran in the integrated irradiation amount that have been calculated at the integrated irradiation amount calculation timings Ta so far, and an addition result of the integrated irradiation amount at the irradiation time estimation timing Tn.

The target imaging time of non-destructive inspection, which is a use case in the present exemplary embodiment, is long, for example, exceeds 30 seconds or one minute. Thus, as the irradiation time estimation timing Tn, a time between several hundred milliseconds (ms) and one second is desirable in consideration of imaging scenes. The time can be optionally set in the radiation imaging system 10, but calculating the prediction value after elapse of sometime improves the prediction accuracy because the number of calculation result samples (the increases Ra) increases and the influence of rising characteristics at start of the radiation irradiation can be reduced. Further, as the increases Ra, which are used to calculate the average increase in the integrated irradiation amount per unit time, the increases Ra after elapse of a predetermined time, excluding those in a rising period of the radiation irradiation are desirably used to predict and calculate the irradiation time, in order to reduce the influence of the rising characteristics at start of the radiation irradiation.

Returning to FIG. 5, in step S503, the drive control unit 400 determines whether to perform a subsequent correction value acquisition operation after the stop of the radiation irradiation in order to correct the electric signals read out by the image readout operation, based on the irradiation time which is the predicted accumulation time. A correction value acquired by execution of the subsequent correction value acquisition operation is referred to as a subsequent correction value. The correction using the subsequent correction value is advantageous in terms of correction performance (a temperature difference, a dark afterimage correction, and signal-to-noise (SN) deterioration), but is low in usability because the waiting time after the imaging is increased by the subsequent correction value acquisition operation. In contrast, the correction using a preliminary correction value acquired in advance by a preliminary correction value acquisition operation (described below) makes the waiting time after the imaging short and is excellent in usability, but is inferior in correction performance in some cases.

Thus, a threshold time for correction method switching determination (hereinafter referred to as a “preset switching time”), which is an optionally preset time, and the irradiation time, which is the accumulation time predicted by the AEC driving, are compared to each other. In a case where the predicted irradiation time is shorter than the preset switching time (YES in step S503), the processing proceeds to step S504. In step S504, the correction using the subsequent correction value is selected. In other words, in step S503, the drive control unit 400 determines whether to cause the readout unit to perform, following the image readout operation (the operation during the period T4), the subsequent correction value acquisition operation during the stop of the radiation irradiation in order to correct the electric signals read out by the image readout operation. More specifically, in a case where the predicted irradiation time as the accumulation time is shorter than the preset switching time, the control unit 225 causes the readout circuit 222 serving as the readout unit to perform the subsequent correction value acquisition operation. Further, the control unit 225 corrects the electric signals read out by the image readout operation, using the subsequent correction value acquired by the subsequent correction value acquisition operation. In the present exemplary embodiment, the subsequent correction value acquisition operation indicates that, following the image readout operation, the readout unit performs an operation of reading out the electric signals from the conversion elements 102 forming the first elements during the stop of the radiation irradiation, thereby acquiring the subsequent correction value.

When the integrated irradiation amount, which is the integrated value of the dose (the reaching dose), reaches the target irradiation dose, then in step S505, a radiation irradiation stop notification is issued. Thereafter, in step S506, the subsequent correction value acquisition operation is performed to acquire the subsequent correction value (also referred to as the subsequent correction image). In step S507, the correction processing (also referred to as the offset correction processing) is performed on the electric signals read out by the image readout operation, using the acquired correction value (image).

In contrast, in a case where the predicted irradiation time (the predicted accumulation time) is longer than the preset time (the preset switching time) (NO in step S503), priority is given to the usability, and the processing proceeds to step S508. In step S508, the correction processing using the preliminary correction value, which is acquired in advance by the preliminary correction value acquisition operation, is selected. The preliminary correction value is acquired in advance by causing the readout unit to perform the preliminary correction value acquisition operation before the radiation irradiation. In other words, before the radiation irradiation, the control unit 225 causes the readout circuit 222 serving as the readout unit to perform the preliminary correction value acquisition operation of acquiring the preliminary correction value for correcting the electric signals read out by the image readout operation, and acquires the preliminary correction value in advance. In the present exemplary embodiment, the preliminary correction value acquisition operation indicates that the readout unit performs an operation of reading out the electric signals from the conversion elements 102 forming the first elements in a state of no radiation irradiation before the radiation irradiation, thereby acquiring the preliminary correction value in advance. When the integrated irradiation amount, which is the integrated value of the reaching dose, reaches the target irradiation dose, then in step S509, the radiation control unit 403 issues a radiation irradiation stop notification.

Thereafter, in step S510, the correction processing (the offset correction processing) using the preliminary correction value (also referred to as the preliminary correction image) acquired in advance is performed on the electric signals read out by the image readout operation. In step S511, the series of imaging operation ends.

The threshold time (the preset switching time) for the correction method switching determination is optionally changeable by a service engineer or the user at the time of installation or based on imaging scenes, and is set based on use applications.

As described above, according to the present exemplary embodiment, in the long-time imaging, the irradiation time is calculated and predicted by the AEC driving during the radiation irradiation, and the method for correcting the electric signals read out by the image readout operation is switched based on the predicted irradiation time. This makes it possible to eliminate presetting of the irradiation time, select the appropriate correction method, and provide the radiation imaging apparatus excellent in usability.

Next, a configuration of the radiation imaging system 10 according to a second exemplary embodiment will be described.

In the present exemplary embodiment, the threshold time (the preset switching time) for the correction method switching determination is set based on an allowable accumulation time (a maximum accumulation time) of one frame in the radiation imaging apparatus 300.

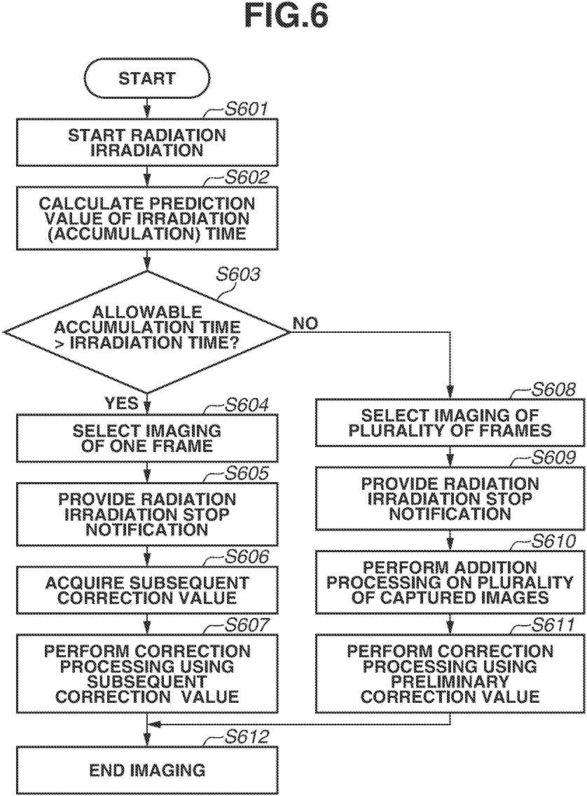

FIG. 6 is a flowchart according to the present exemplary embodiment. In step S601, the radiation irradiation is started by the user’s operation. In step S602, the drive control unit 400 performs the AEC driving of the radiation imaging apparatus 300 to calculate the integrated irradiation amount, which is the integrated value of the reaching dose, using the irradiation time prediction unit 408 during the imaging. As described above, the irradiation time prediction unit 408 calculates the increase in the integrated irradiation amount per constant unit time, and predicts and calculates, based on a separately set target irradiation dose, the accumulation time (the irradiation time) taken to reach the target irradiation dose.

In step S603, the drive control unit 400 compares the accumulation time (the irradiation time) predicted by the AEC driving during the imaging, with the allowable accumulation time. The allowable accumulation time is stored in the memory 402 of the control unit 225. In a case where the predicted accumulation time is shorter than the allowable accumulation time (YES in step S603), the processing proceeds to step S604. In step S604, imaging of one frame is selected and performed. In other words, the control unit 225 includes the accumulation time acquisition unit that acquires information about the charge accumulation time based on the electric signals read out by the dose detection operation, and determines the number of times of the image readout operation based on the information about the charge accumulation time. As described above, in a case where the accumulation time is shorter than the preset allowable accumulation time, the control unit 225 determines the number of times of the image readout operation to be one, and performs the imaging of one frame.

As the image correction method at that time, the subsequent correction value acquisition operation may be used and performed to acquire the subsequent correction value in step S606 in a manner similar to step S506, and the correction may be performed using the acquired subsequent correction value. Alternatively, the correction processing may be performed by using the preliminary correction value acquired in advance. Processing in steps S605, S607, and S612 is similar to the above-described processing in steps S505, S507, and S511, respectively. Thus, descriptions thereof will be omitted.

In contrast, in a case where the accumulation time is longer than the preset allowable accumulation time (NO in step S603), the processing proceeds to step S608. In step S608, the control unit 225 determines the number of times of the image readout operation to two or more. When two or more times of the image readout operation is selected (determined), namely, when imaging of a plurality of images is selected, then in step S610, addition processing is performed on the captured images after the stop of the radiation irradiation. The accumulation time in the case of the imaging of the plurality of frames may include a desired one pattern or a plurality of patterns. In a case where the preliminary correction value is used for the correction processing thereafter, the preliminary correction values (the preliminary correction images) corresponding to the plurality of patterns of the accumulation time are to be acquired in advance. However, as the number of added images increases in the radiation imaging, noise components are also added to cause SN deterioration. Thus, with respect to the predicted accumulation (irradiation) time, the accumulation time that can reduce the number of images to be added and shorten the waiting time after the stop of the radiation irradiation is desirably selected among the plurality of patterns.

In particular, in the case of the imaging of the plurality of frames, the timing for the radiation control unit 403 to issue a radiation irradiation stop instruction is to be paid attention to. Overlapping of the radiation irradiation timing with the period of the image readout operation of reading out the electric signals from the conversion elements 102 causes an artifact such as shading in the image. To prevent the artifact, the radiation control unit 403 having a radiation stop instruction function controls, in the imaging of a final one of the plurality of frames, the timing for issuing the radiation irradiation stop instruction to the radiation generation apparatus 324 so that the radiation irradiation ends during the accumulation period without overlapping with the period of the image readout operation. As described above, the control unit 225 has the stop instruction function of issuing the radiation irradiation stop instruction based on the electric signals read out by the dose detection operation, and performs control to issue the stop instruction in a period during which the readout unit does not perform the image readout operation. Processing in steps S609 and S611 are similar to the processing in steps S509 and S510, respectively. Thus, descriptions thereof will be omitted.

As described above, in the present exemplary embodiment, in the long-time imaging, the irradiation time is predicted by the AEC driving during the irradiation, and the number of frames to be captured is changed based on the maximum accumulation time of one frame in the radiation imaging apparatus. This makes it possible to provide the radiation imaging apparatus excellent in usability.

Although the exemplary embodiments of the present disclosure have been described in detail above, the present disclosure is not limited to such specific exemplary embodiments, and can be modified and changed in various manners within the scope of the spirit of the present disclosure according to the claims.

The exemplary embodiments of the present disclosure can be implemented by supplying a program for implementing one or more functions according to the above-described exemplary embodiments to a system or an apparatus via a network or a storage medium, and causing one or more processors in a computer of the system or the apparatus to read out and execute the program. Further, the exemplary embodiments of the present disclosure can be implemented by a circuit (e.g., an application specific integrated circuit (ASIC)) for implementing one or more functions according to the exemplary embodiments.

According to the exemplary embodiments of the present disclosure, in the radiation imaging in which the radiation irradiation time, namely, the charge accumulation time based on the radiation irradiation is variable, it is possible to improve the image quality of captured images and the usability of the imaging apparatus.

### Other Embodiments

Embodiment(s) of the present disclosure can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present disclosure has been described with reference to exemplary embodiments, it is to be understood that the disclosure is not limited to the disclosed exemplary embodiments. The scope of the following claims is to be accorded the broadest interpretation so as to encompass all such modifications and equivalent structures and functions.

This application claims the benefit of Japanese Patent Application No. 2021-191525, filed Nov. 25, 2021, which is hereby incorporated by reference herein in its entirety.

What is claimed is:

1. A radiation imaging apparatus comprising:
an element portion including a first element and a second element each configured to output an electric signal based on charge accumulated in response to radiation irradiation;
a readout unit configured to read out the electric signal; and
a control unit configured to control an operation of the readout unit so that the readout unit performs a dose detection operation of reading out the electric signal from the second element during the radiation irradiation, and performs an image readout operation of reading out the electric signal from the first element after stop of the radiation irradiation,
wherein the control unit includes an accumulation time acquisition unit configured to acquire information about an accumulation time of the charge based on the electric signal read out by the dose detection operation, and determines, based on the information about the accumulation time, whether to cause the readout unit to perform, following the image readout operation, a subsequent correction value acquisition operation for correcting the electric signal read out by the image readout operation during the stop of the radiation irradiation.

2. The radiation imaging apparatus according to claim 1, wherein, as the subsequent correction value acquisition operation, the readout unit performs an operation of reading out the electric signal from the first element during the stop of the radiation irradiation, following the image readout operation.

3. The radiation imaging apparatus according to claim 2, wherein, in a case where the accumulation time is shorter than a preset time, the control unit causes the readout unit to perform the subsequent correction value acquisition operation, and corrects the electric signal read out by the image readout operation by using a correction value acquired by the subsequent correction value acquisition operation.

4. The radiation imaging apparatus according to claim 1, wherein, before the radiation irradiation, the control unit causes the readout unit to perform a preliminary correction value acquisition operation of acquiring a preliminary correction value for correcting the electric signal read out by the image readout operation.

5. The radiation imaging apparatus according to claim 4, wherein, as the preliminary correction value acquisition operation, the readout unit performs an operation of reading out the electric signal from the first element in a state of no radiation irradiation before the radiation irradiation.

6. The radiation imaging apparatus according to claim 5, wherein, in a case where the accumulation time is longer than a preset time, the control unit corrects the electric signal read out by the image readout operation by using the preliminary correction value acquired by the preliminary correction value acquisition operation.

7. A radiation imaging apparatus comprising:
an element portion including a first element and a second element each configured to output an electric signal based on charge accumulated in response to radiation irradiation;
a readout unit configured to read out the electric signal; and
a control unit configured to control an operation of the readout unit so that the readout unit performs an image readout operation of reading out the electric signal from the first element, and performs a dose detection operation of reading out the electric signal from the second element during the radiation irradiation,
wherein the control unit includes an accumulation time acquisition unit configured to acquire information about an accumulation time of the charge based on the electric signal read out by the dose detection operation, and determines a number of times of the image readout operation based on the information about the accumulation time.

8. The radiation imaging apparatus according to claim 7, wherein, in a case where the accumulation time is shorter than a preset allowable accumulation time, the control unit determines the number of times of the image readout operation to be one.

9. The radiation imaging apparatus according to claim 7, wherein, in a case where the accumulation time is longer than a preset allowable accumulation time, the control unit determines the number of times of the image readout operation to be two or more.

10. The radiation imaging apparatus according to claim 9, wherein the control unit includes a stop instruction function of issuing a stop instruction to stop the radiation irradiation based on the electric signal read out by the dose detection operation, and performs control to issue the stop instruction in a period during which the readout unit does not perform the image readout operation.

11. A radiation imaging apparatus comprising:
an element portion including a first element and a second element each configured to output an electric signal;
a readout unit configured to read out the electric signal; and
a control unit configured to control an operation of the readout unit so that the readout unit performs a first operation of reading out the electric signal from the second element during the radiation irradiation, performs a second operation of reading out the electric signal from the first element following the first operation, and performs a third operation of reading out the electric signal from the first element during stop of the radiation irradiation following the second operation,
wherein the control unit includes an irradiation time acquisition unit configured to acquire information about an irradiation time of the radiation based on the electric signal read out by the first operation, and determines whether to cause the readout unit to perform the third operation based on the information about the irradiation time.
